(19) United States
(12) Patent Application Publication
(10) Pub. No.: US 2013/0091932 A1
(43) Pub. Date: Apr. 18, 2013

(54) Process and Method for Broad Spectrum Amino Acid Challenged Urinary Organic Acids

(71) Applicants: James Alexander Brown, III, Johns Creek, GA (US); Richard S. Lord, Waleska, GA (US)

(72) Inventors: James Alexander Brown, III, Johns Creek, GA (US); Richard S. Lord, Waleska, GA (US)

(21) Appl. No.: 13/652,375

(22) Filed: Oct. 15, 2012

Related U.S. Application Data
(60) Provisional application No. 61/546,808, filed on Oct. 13, 2011.

(51) Int. Cl. G01N 33/50

(52) U.S. Cl. CPC G01N 33/50
USPC 73/61.41; 426/656

(57) ABSTRACT

Discloses is a process for detecting and evaluating metabolic disturbances and specific nutrient insufficiencies within an individual. The bio-chemical pathways of the individual are challenged by consuming an oral amino acid supplement prior to urine sample collection. The oral amino acid supplement includes at least two of the amino acids selected from the group: L-Valine, L-Leucine, L-Isoleucine, L-Tryptophan, L-Histidine, and L-Alanine. After processing the amino acid supplement, a urine sample is collected and a urinary organic acid profile is evaluated. The levels of metabolic markers within the organic acid profile are evaluated to determine the presence of metabolic disturbances, nutrient insufficiencies, genetic impairments, toxicant impairments, mitochondrial function impairment, or small intestinal dysbiosis, within the individual.

### CROSS REFERENCE TO A PROVISIONAL APPLICATION

This application claims the benefit of Provisional Application Ser. No. 61/546,808, filed on Oct. 13, 2011, the entirety of which is hereby incorporated herein by reference.

### BACKGROUND OF THE INVENTION

1. Field of the Invention

The present invention relates to the use of an amino acid challenge to evaluate specific nutrient deficiencies within an individual. The amino acid challenge elevates urinary metabolic markers to explore specific nutrient deficiencies. Precursor amino acids are administered orally to increase the metabolic and bio-chemical pathway flux, thereby enhancing the excreted metabolic marker concentrations.

2. Background of the Invention

The following literature is of use in the subject matter of the present invention and is incorporated herein by reference:
1. Knapp, A., [Tryptophan loading and vitamin B6 deficiency. I. Excretion of xanthurenic acid, kynurenine, trigonelline amide, and 5-hydroxyindoleacetic acid in various diseases.]. Dtsch Gesundheitsw, 1961. 16: p. 941-51.
2. Abbassy, A. S., M. M. Zeitoun, and M. H. Abouiwfa, The state of vitamin B6 deficiency as measured by urinary xanthurenic acid. J Trop Pediatr, 1959. 5: p. 45-50.
3. Mock, D. M., et al., Biotin status assessed longitudinally in pregnant women. J Nutr, 1997. 127(5): p. 710-6.
4. Gijsman, H. J., et al., A dose-finding study on the effects of branch chain amino acids on surrogate markers of brain dopamine function. Psychopharmacology (Berl), 2002. 160(2): p. 192-7.
5. Schuller, E., [Folic Acid Metabolism and the Figlu Test]. Presse Med, 1965. 73: p. 1411-4.
6. Roon-Djordjevic, B. v. and S. Cerfontain-van, Urinary excretion of histidine metabolites as an indication for folic acid and vitamin B12 deficiency. Clin Chim Acta, 1972. 41: p. 55-65.
7. Mock, D. M., et al., Indicators of marginal biotin deficiency and repletion in humans: validation of 3-hydroxyisovaleric acid excretion and a leucine challenge. Am J Clin Nutr, 2002. 76(5): p. 1061-8.
8. Mock, N., et al., Increased urinary excretion of 3-hydroxyisovaleric acid and decreased urinary excretion of biotin are sensitive early indicators of decreased biotin status in experimental biotin deficiency. Am J Clin Nutr, 1997. 65: p. 951-8.
9. Mock, D. M., et al., 3-Hydroxypropionic acid and methylcitric acid are not reliable indicators of marginal biotin deficiency in humans. J Nutr, 2004. 134(2): p. 317-20.
10. S., L. R., et al., Weekly Biological Variability of Urinary Organic Acids. North American Journal of Medicine and Science, 2012. 5(3): p. 9.

Multiple studies have demonstrated the usefulness of urinary metabolic markers that become elevated in specific nutrient deficiencies. Precursor amino acids sometimes are administered to increase the pathway flux, thereby enhancing the excreted marker concentrations. [1-3]

Adoption of challenge procedures in routine clinical laboratory testing requires consideration of safety for dosages used. For comparison purposes, it is useful to consider that a typical adult can easily consume more than 25 grams of protein in a single meal that typically would deliver the equivalent mass of amino acids to portal blood over an interval of 2 hours. Branched-chain amino acids (BCAA) were well-tolerated at up to 60 g/day in a dose-finding study for suppression of brain dopamine output. [4] The development of urinary formiminoglutamic acid as a marker of folic acid deficiency was based on human studies where up to 15 g of L-histidine was administered safely.[5, 6] Similarly, a 3 to 5 g bolus of L-tryptophan was administered to humans in studies that led to the proposal of xanthurenic acid elevation as a biochemical marker of vitamin B6 deficiency.[1, 2] The biotin deficiency marker, 3-hydroxyisovalerate was developed by administration of 15 g of isoleucine to humans.[3, 7-9] Even though the dosages were higher than those used in this study, no adverse effects were reported by any subjects.

The prior art reference noted herein describe the use of a single amino acid administered to a patient followed by evaluation of the patients urinary organic acid profile. In the prior art, the patient is stressed by a substantial dose of an individual amino acid, the amino acid is processed via the metabolic pathways and the results reflected in the urinary acid profile. As with any multivariable complex system, the results of using a single amino acid to stress the metabolic and biochemical pathways of the individual may be studied, but the resulting urinary organic acid profile is incomplete.

What is absent in the prior art is a broad spectrum amino acid challenge which stimulates the metabolic pathways across a range of amino types and explores the reaction differences of the individual to broad spectrum stress of the metabolic pathways as opposed to stressing the metabolic pathways with a single amino acid type. Due to the interdependence of the metabolic pathways within the individual, the broad spectrum amino acid challenge will have different results than those predicted by a single amino acid challenge.

Considering the aforementioned, there is an obvious need in the art for a process and method using a broad spectrum amino acid challenge which stimulates the metabolic pathways across a range of amino types. The broad spectrum amino acid challenge would stimulate the metabolic and biochemical pathways and expose the reaction differences of the individual to broad spectrum stress of the metabolic pathways. The efficacy of the amino acid challenge to stress test the metabolic and biochemical pathways of the individual are positively affected by stimulating the metabolic pathways using a broad spectrum of amino acids. It is to such a broad spectrum amino acid challenge process and method that the present invention is primarily directed.

### SUMMARY OF THE INVENTION

In one aspect the present invention provides a process for a process for detecting and evaluating metabolic disturbances and specific nutrient insufficiencies within an individual. The individual consumes an oral amino acid challenge supplement comprising at least two of the amino acids selected from the group: L-Valine, L-Leucine, L-Isoleucine, L-Tryptophan, L-Histidine, and L-Alanine. A urinary sample is collected from the individual after consuming the oral amino acid supplement and giving sufficient time for the amino acids to move thru the individuals bio-chemical pathways. A urinary sample is then evaluated to obtain an organic acid profile. The levels of metabolic markers within the organic acid profile are evaluated to determine the presence of metabolic disturbances or nutrient insufficiencies within the individual.

The levels of metabolic markers within the urinary sample are evaluated to determine any genetic impairments, toxicant impairments, mitochondrial function impairment, or small intestinal dysbiosis within the individual. The individual is challenged with the oral amino acid challenge supplement at least 8 hours prior to collection of the urinary sample, for example, prior to 11 PM one evening and the collection of the urinary sample occurring after 7 AM of the following morning.

When the individual weighs between 100 to 200 lbs, the dosage of the amino acids within the oral amino acid challenge supplement comprise at least one of: 2.4-3.6 grams L-Valine, 3.1-4.7 grams L-Leucine, 3.1-4.7 grams L-Isoleucine, 1.2-1.8 grams L-Tryptophan, 3.6-5.4 grams L-Histidine, or 2.4-3.6 grams L-Alanine. In another aspect, the dosage of the amino acids within the oral amino acid challenge supplement comprise at least one of: 3.0 grams L-Valine, 3.9 grams L-Leucine, 3.9 grams L-Isoleucine, 1.5 grams L-Tryptophan, 4.5 grams L-Histidine, or 3.0 grams L-Alanine.

When the individual weighs more than 200 lbs, the dosage of the amino acids within the oral amino acid challenge supplement comprise at least one of: 3.2-4.8 grams L-Valine, 4.1-6.2 grams L-Leucine, 4.1-6.2 grams L-Isoleucine, 1.6-2.4 grams L-Tryptophan, 4.8-7.2 grams L-Histidine, or 3.2-4.8 grams L-Alanine. In another aspect, the dosage of the amino acids within the oral amino acid challenge supplement comprise at least one of: 4.0 grams L-Valine, 5.2 grams L-Leucine, 5.2 grams L-Isoleucine, 2.0 grams L-Tryptophan, 6.0 grams L-Histidine, or 4.0 grams L-Alanine.

When the individual weighs between 50 to 100 lbs, the dosage of the amino acids within the oral amino acid challenge supplement comprise at least one of: 1.6-2.4 grams L-Valine, 2.1-3.1 grams L-Leucine, 2.1-3.1 grams L-Isoleucine, 0.8-1.2 grams L-Tryptophan, 2.4-3.6 grams L-Histidine, or 1.6-2.4 grams L-Alanine. In another aspect, the dosage of the amino acids within the oral amino acid challenge supplement comprise at least one of: 2.0 grams L-Valine, 2.6 grams L-Leucine, 2.6 grams L-Isoleucine, 1.0 grams L-Tryptophan, 3.0 grams L-Histidine, or 2.0 grams L-Alanine.

In another aspect, an oral amino acid challenge supplement comprises at least two of the amino acids selected from the group: L-Valine, L-Leucine, L-Isoleucine, L-Tryptophan, L-Histidine, and L-Alanine. The oral amino acid challenge supplement is consumed by an individual and processed by the individuals bio-chemical pathways prior to a urine sample collection. The urine sample collected is then used to evaluate a urinary organic acid profile. The levels of metabolic markers within the urinary organic acid profile are evaluated to determine the presence of metabolic disturbances or nutrient insufficiencies within the individual.

These and other aspects of the invention will become apparent from the following description of the preferred embodiments taken in conjunction with the tables and figures. As would be obvious to one skilled in the art, many variations and modifications of the invention may be effected without departing from the spirit and scope of the novel concepts of the disclosure.

### BRIEF DESCRIPTION OF THE TABLES AND FIGURES

Table 1 summarizes the amino acids included in the challenge formula, urinary metabolic markers affected and related nutrients or metabolic effects.

Table 2 shows the composition of amino acid formula used for a 3 person study.

Table 3 shows the composition of amino acid formula used for a 38 person study.

FIG. 1 shows the range of urinary FIGLU changes with amino acid challenge containing L-histidine.

FIG. 2 shows the results of the 3 person study for all analytes.

FIG. 3 shows the results of the 38 person study for all analytes.

### DETAILED DESCRIPTION OF PREFERRED EMBODIMENTS

The present invention stimulates across a range of amino types and explores the reaction differences of the individual/patient to broad spectrum stress of the metabolic pathways as opposed to stressing the metabolic pathways with a single amino acid type. The invention also explores the interdependence of the metabolic and biochemical pathways within the individual when subjected to a broad spectrum amino acid challenge. Due to the interdependence of the metabolic pathways within the individual, the broad spectrum amino acid challenge will have different results than those predicted by a single amino acid challenge. The efficacy of the amino acid challenge to stress test the metabolic and biochemical pathways of the individual are positively affected by stimulating the metabolic pathways using a broad spectrum of amino acids.

In the present invention, the degree of increases in measured compounds that are produced by the challenge is determined by the capacity of an individual to clear the metabolic intermediates so that urinary excretion is not elevated. Even mild insufficiencies or enzyme abnormalities that might be missed under non-challenged conditions may become apparent under conditions of the challenge. Individuals with excellent nutrient repletion status are revealed by the fact that no elevations of organic acids are found, even though the pathways were challenged to process higher levels of amino acid substrates and metabolic pathway intermediates.

The sensitivity and reliability of detecting metabolic disturbances by finding elevated levels of urinary organic acids is improved when the subject consumes an appropriate dose of precursor amino acids preceding urine collection. During the sleep period, the amino acids flow through metabolic pathways where accumulation of specific intermediates that spill into urine at elevated levels can indicate nutrient deficiencies and genetic or toxicant impairments. This study examined the effects of dosing with a broad spectrum of free-form amino acids to generate flux through multiple biochemical pathways that contain markers of vitamin deficiencies.

An initial pilot study was conducted using 3 subjects. A second study was conducted using 38 adult subjects who were chosen as having no diagnosed diseases and normal work habits. In the 3-person study, 2 healthy males with ages of 65 years (Subject 1) and 55 years (Subject 2) and one healthy 56 year old female (Subject 3) followed the protocol. In the 38-subject study, healthy female (18) and male (20) subjects were recruited. On two consecutive nights urine was collected for performing analysis of 46 compounds included in a profile of organic acids. On the second night, a single bolus of crystalline pure L-amino acids was consumed dissolved in water. All amino acids used were L-stereoisomers unless designated otherwise by the “D-”prefix. In the 3-person pilot, dosing included tyrosine, valine, leucine, isoleucine, histidine, and tryptophan as free-form or chloride salts at a total load of approximately 400 mg/kg body weight. For the 38-subject study, the dosing formula included a similar mixture containing alanine, valine, leucine, isoleucine, histidine, and tryptophan. Table 1 summarizes the amino acids included in the challenge formula, urinary metabolic markers affected and related nutrients or metabolic effects.

The Amino acid challenge dose protocol was as follows. For 2 consecutive nights, participants collected overnight urine specimens. On the second night, before going to bed, they took approximately 400 mg/kg body weight of the challenge dose of amino acids. The doses were supplied in packets containing 6.6 g of the mixture, and the number of packets was adjusted from 2 to 4 according to body weight that exceeded 100, 150 and 200 lbs, respectively. Thus the dose administration was simplified, while maintaining a dosage/kg body weight in the range of +−16% of the median body weight for each increment. For a 150 lb individual, the formula supplied the amounts shown in Table 1. The amino acid blend was dissolved in water and consumed all at once. The first night's specimen was frozen and delivered to the laboratory with the frozen second night's specimen.

In the 3-person study, there were mild complications of varying severity apparently arising from stimulation of catecholamine synthesis by the inclusion of tyrosine. The results confirmed both the catecholamine stimulation and multiple other enhancements of organic acid levels. In the 38-person study, compared with the non-challenged urine, the challenged specimens showed higher average concentrations of metabolic markers including branched-chain keto acids, 3-hydroxyisovaleric, formiminoglutamic (FIGLU), and xanthurenic acids. These metabolic marker compounds become elevated due to deficiencies of thiamin, biotin, folate, and vitamin B6 respectively. Table 2 shows the composition of amino acid formula used for 3-person study. Table 3 shows the composition of amino acid formula used for 38-person study.

There was a high degree of variability in levels of metabolic markers among subjects. The added L-alanine apparently led to increased levels of pyruvate, succinate, fumarate and malate in some subjects. These changes indicate the presence of specific points of biochemical individuality most likely related to weakness in mitochondrial oxidation pathways. In addition, some subject showed large increases in various other amino acid metabolites indicating other types of specific metabolic states, including stimulation of intestinal microbial growth.

Both formulas of amino acids used as a single dose challenge in the 3 subject and 38 subject studies successfully produced elevations in multiple metabolic markers of nutrient deficiencies. In a few of the otherwise normal subjects, strong increases were found, indicating latent deficiency conditions that would not be detected by performing unchallenged organic acid analysis. The broad spectrum amino acid challenge procedure greatly enhances the clinical sensitivity for detecting deficiencies of the relevant vitamins, and it increases the ability to detect individualized states of mitochondrial function impairment and small intestinal dysbiosis.

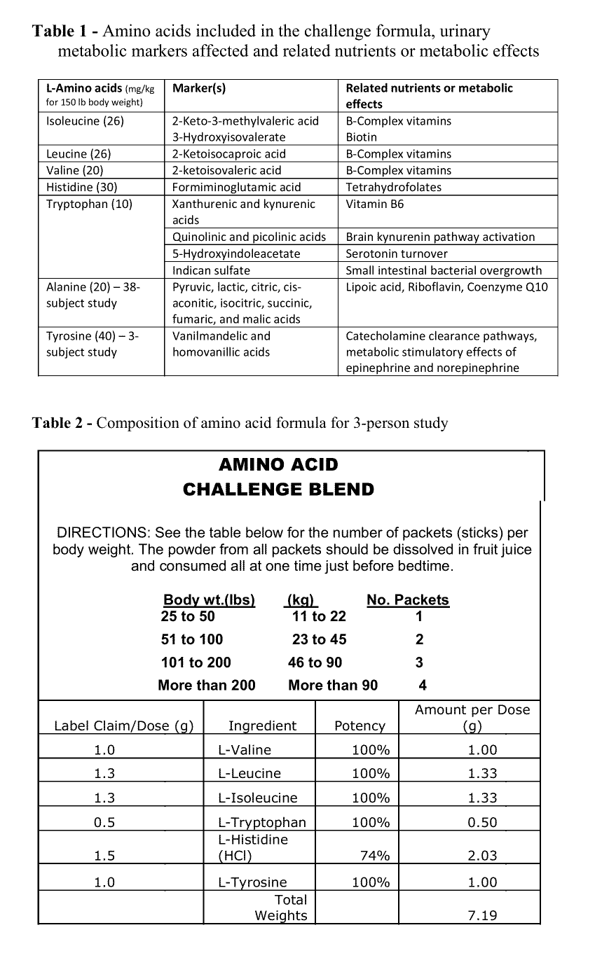

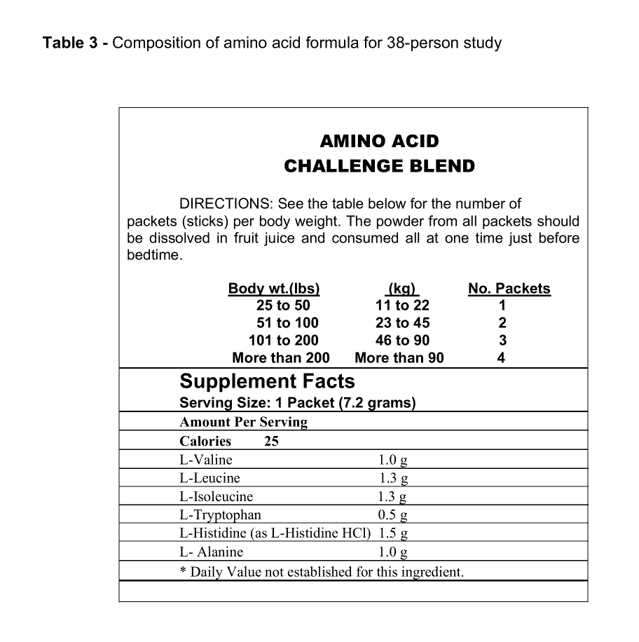

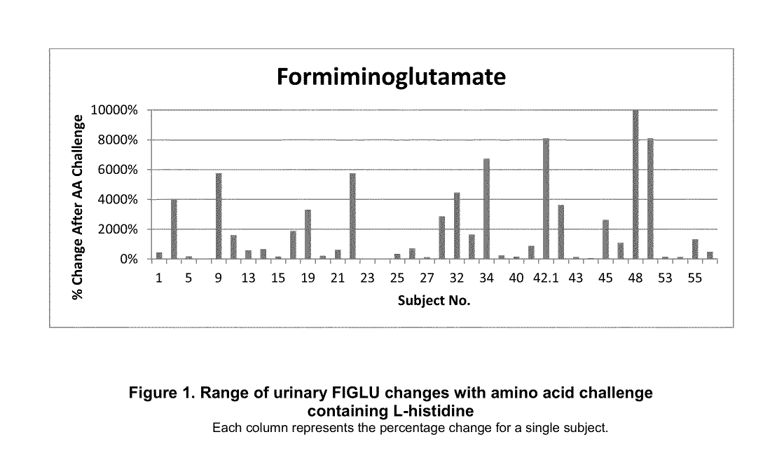

Although output of many of the organic acids was increased by the challenge, not all individuals showed higher levels of metabolic markers, even for the compounds that displayed the greatest average increase. This result demonstrates that, even with the extra challenge, some individuals have sufficient capacity to clear the intermediates. Some individuals had a tendency to show lower post-challenge metabolic marker levels for several compounds, indicating a general catabolic stimulation by the challenge. The average post-challenge increase for all analytes over all subjects was 130%, with individual changes ranging from 6 to 2000%. The pathways most dramatically affected where those for catabolism of histidine and tryptophan. FIG. 1 shows that 12 of the 38 subjects (32%) had increases of more than 2000% above unchallenged output for the histidine pathway intermediate FIGLU. Those individuals are at risk of deficiency for dietary folates. For xanthurenic acid, 42% of the subjects had levels that increased by more than 700%, demonstrating risk of vitamin B6 deficiency.

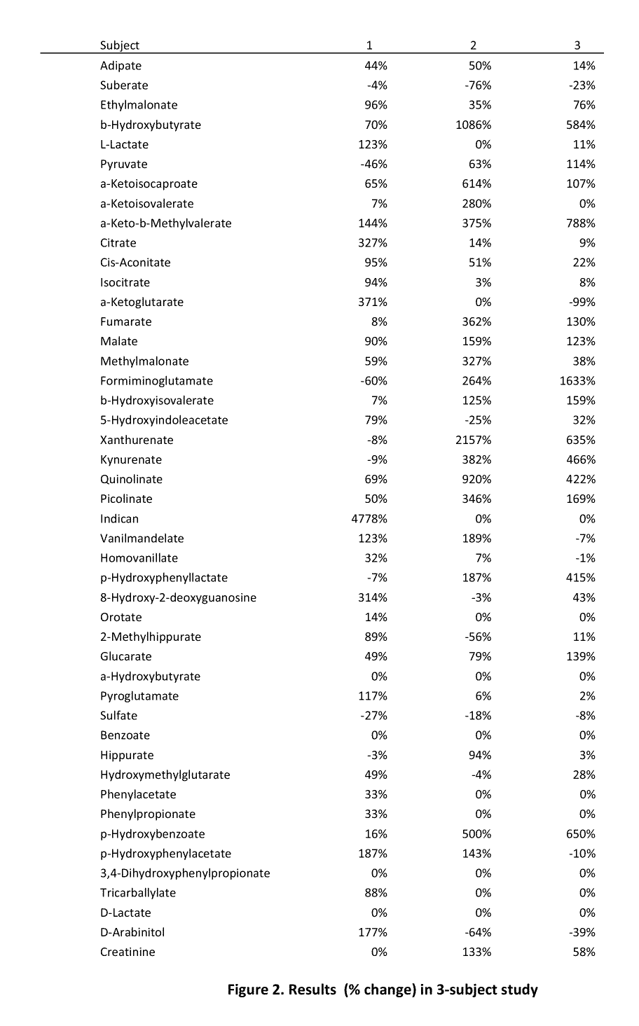

FIG. 2 shows the results of the 3 person study for all analytes. The increased flux of carbon fragments from all of the amino acids in the challenge dose produced various degrees of ketosis, as shown by the changes of 3-hydroxybutyrate that ranged from −90% to 575%. Differences in insulin response to stimulate uptake of amino acids across cell membranes are likely part of the explanation for this variability.

As seen in FIG. 2, the branched chain amino acids produced post challenge increases greater than 100% for their keto acid products 2-keto-3-methylvaleric, 2-ketoisocaproate and 2-ketoisovalerate in 50, 18 and 11% of subjects, respectively. This may be due to variations in stores of B-complex vitamins and individual polymorphisms in branched-chain keto acid dehydrogenase.

One reason for including tyrosine in the challenge formula was to see the effect on fumarate that is released from its catabolism. Subjects 2 and 3 showed rises in fumarate and corresponding increases for the next product, succinate in the cyclic pathway. However, subject 1 had slower release of fumarate from tyrosine, and he displayed a tendency for the intermediates, malate through isocitrate to become elevated. This difference is further reflected in the flow of tyrosine through competing pathways. The catabolic end product, vanilmandelate, from the catecholamine pathway product, epinephrine, was elevated in Subject 1 who experienced an extended stimulation of heart rate following ingesting the amino acid mixture.

FIG. 2 also depicts the results for B-Vitamin insufficiency markers. Since the three subjects may be presumed to be in better than average nutritional status, increases in concentrations of deficiency biomarker compounds are due to substrate overload of the cofactor-saturated enzymes rather than nutritional deficiencies in the B-complex vitamins. Thus the experiment clearly shows that higher normal limits are needed for reporting levels on ffAA-challenge profiles. Of interest is that the three subjects displayed marked differences. Subject 1 had insignificant changes, and his level of FiGLU actually appeared to drop slightly after the challenge, while Subject 3 showed a 1500% increase. A larger population was required to determine how to set amino acid-challenged reference ranges for these compounds.

In FIG. 2, the largest magnitude of changes was found in the metabolites of tryptophan compounds, and a great deal of variability in dominant pathways among the subjects is apparent. The lowest magnitude of change and the least variability is seen in the monoamine pathway to serotonin and 5-hydroxyindoleacetic acid. Multiple reports have concluded that the kynurenin pathway in hepatic and brain tissues is the most active route of disposition for any excess of tryptophan, and great activity was indicated by these data. Subject 1 showed nil changes in the vitamin B6-dependent intermediates, xanthurenate and kynurenate, while subjects 2 and 3 showed pronounced increases with the tryptophan challenge. The subsequent intermediates from the kynurenin pathway, quinolinate and picolinate were also much more greatly increased in Subjects 2 and 3 than in Subject 1. Among the competing pathways of monoamine and kynurenin formation, only Subject 1 showed a slight preference in the monoamine direction. Meanwhile, the amount of tryptophan available for cellular processing in Subject 1 apparently was lowered by a high rate of intestinal bacterial conversion to indole, as evidenced by his great increase in the excretory product, indican. This can explain the generally smaller increases in kynurenin pathway products.

As the data in FIG. 2 presents, catecholamine a marker of epinephrine, norepinephrine and dopamine synthesis is affected. The origin of tachycardia and sleep disturbance reported by Subjects 1 and 3 may be ascribed as the outcome of increased epinephrine output according to the significant rise in levels of vanylmandelate, a marker of norepinephrine and epinephrine. Subject 2, who reported no such effects, correspondingly shows no change in VMA. Under the conditions of this experiment, there is little apparent shift in output of dopamine, suggesting efficient conversion to norepinephrine and epinephrine.

For the compounds relating to cell division and oxidative stress, p-hydroxyphenyllactate (HPLA) and 8-hydroxy-2′-deoxyguanine, a large divergence of changes was found again among the subjects. Subject 1 showed nil changes in HPLA, while, quite surprisingly, Subjects 2 and 3 showed large increases. The only known role of HPLA involves its activity in regulation of cell division rates, and the data shows a moderate challenge of amino acids exerts such a strong stimulatory effect on the pathway. The stimulation of heart ouput and thus of systemic metabolism explains the particularly great increase seen for the DNA repair product, 8-hydroxy-2′-deoxyguanosine (8OHdG).

The time scale used in the experiment was short compared to time required for the full sequence of increased ROS production, followed by increased DNA damage and subsequent DNA repair in an individual. The data presents that the few hours of increased cardiac activity stimulated only the final aspect of DNA repair. The conclusion is supported by the finding of significantly higher levels of 8OHdG in people who practice well-managed caloric restriction. The data presents that individuals maintain a background level of unrepaired DNA damage which, under the appropriate metabolic circumstances can be lowered by greater rates of repair activity.

Only relatively small changes were found in analytes associated with toxicant exposure and detoxification. The result is consistent with the low toxic potential of ffAA. All 3 subjects showed a slight increase in glucarate, suggesting that general hepatic detox activity as increased, illustrating the ability of amino acids to stimulate Phase 1 and 2 detoxification systems.

The changes found in intestinal microbial products were unexpected because the high efficiency for ffAA absorption in the upper small intestine might not allow time for significant bacterial uptake and degradation. One explanation is that the sudden and strong rise in circulating free amino acid concentrations stimulates unusual activity in hepatic or enterocyte pathways that might lead to the products normally thought to arise from intestinal bacterial metabolism. However, the large increase for indican described above for the metabolites of tryptophan is very unlikely to have arisen from such human tissue metabolism. Thus the data shows that upper jejunal bacteria have the capacity to achieve such activity. This type of challenge testing can reveal distinguishing features of those bacterial communities in an individual. Of note, the absence of indican changes for Subjects 2 and 3 while those individuals showed further differential features of p-hydroxybenzoate (both of these subjects) and p-hydroxyphenylacetate (subject 2) elevation. And, for these three subjects, distinctly different patterns may be defined utilizing only three compounds derived from bacterial metabolism.

The data also shows negative changes in D-arabinitol in Subjects 2 and 3 with a significant positive shift for Subject 1. These differences suggest that Subject 1 may maintain significantly greater levels of upper jejunal intestinal yeast than Subjects 2 and 3. Thus the invention presents the possibility of detecting much greater changes in patients who have symptoms consistent with yeast overgrowth.

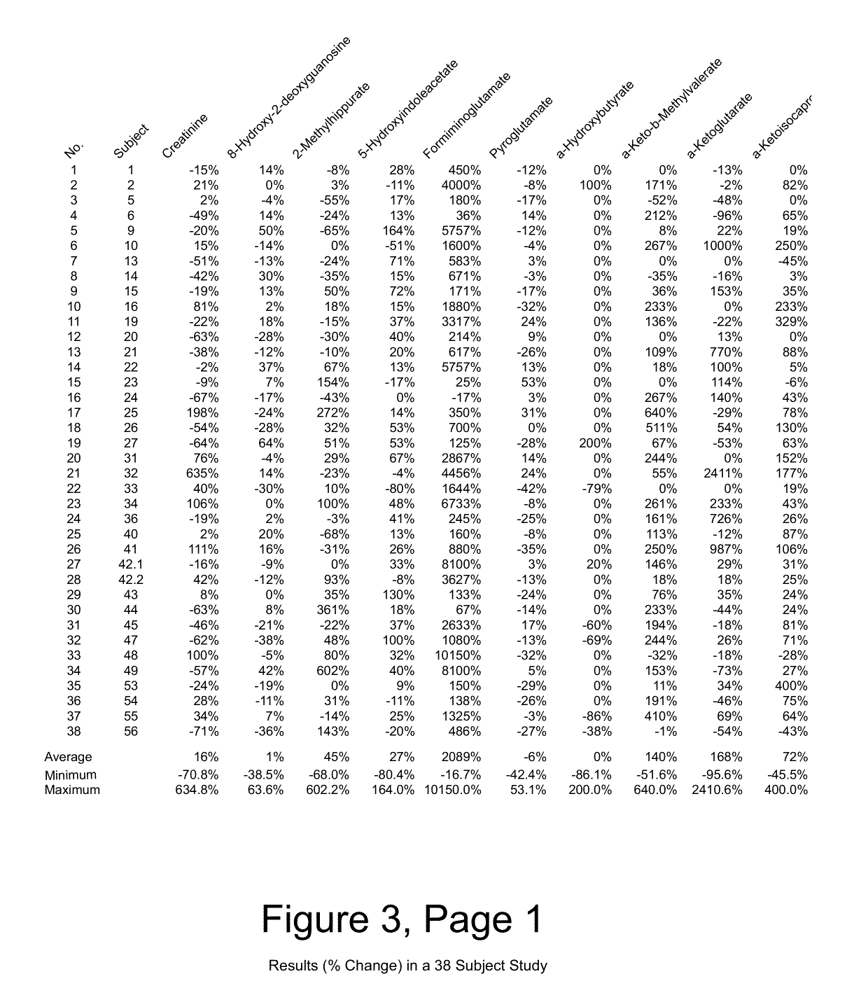

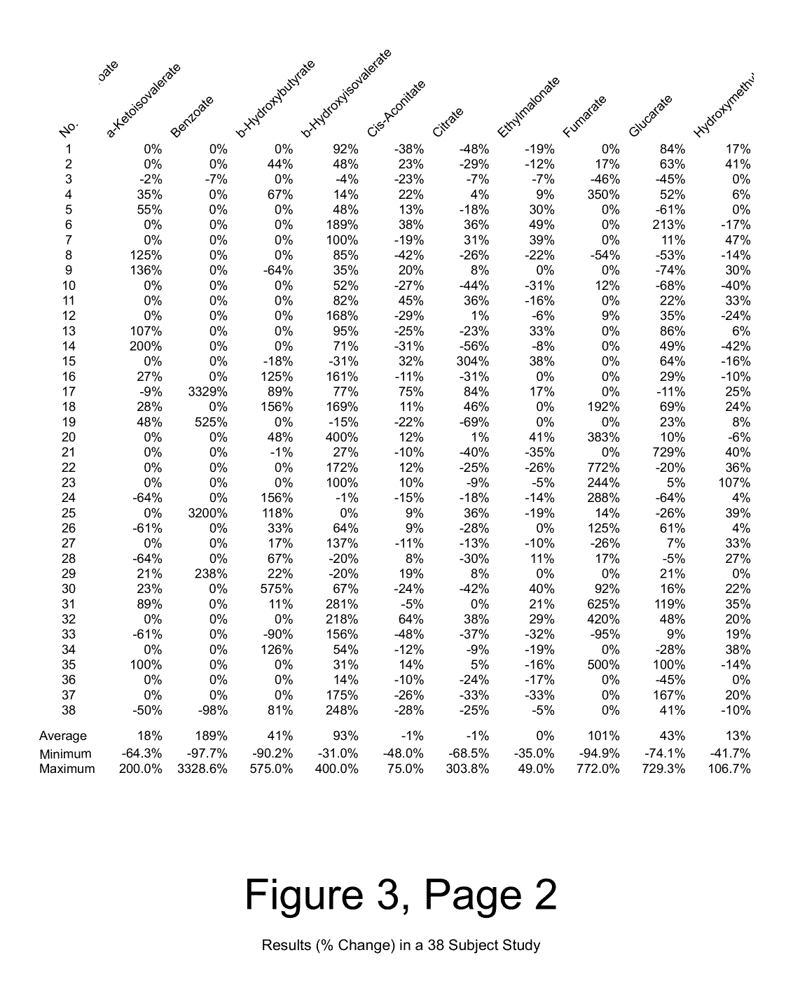

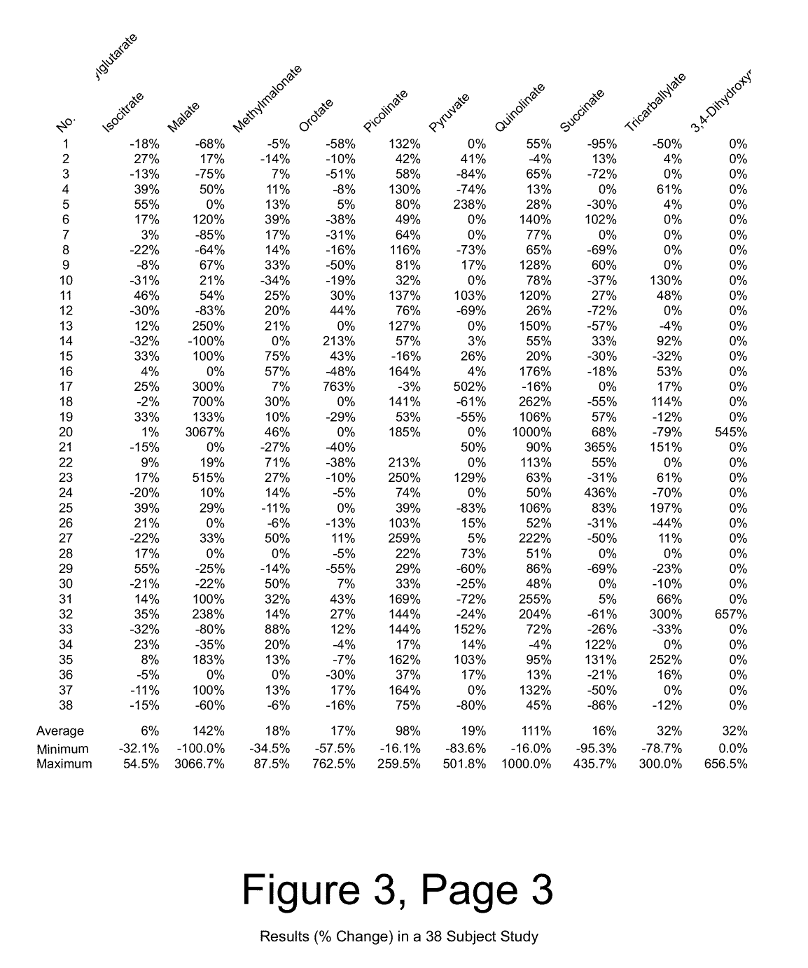

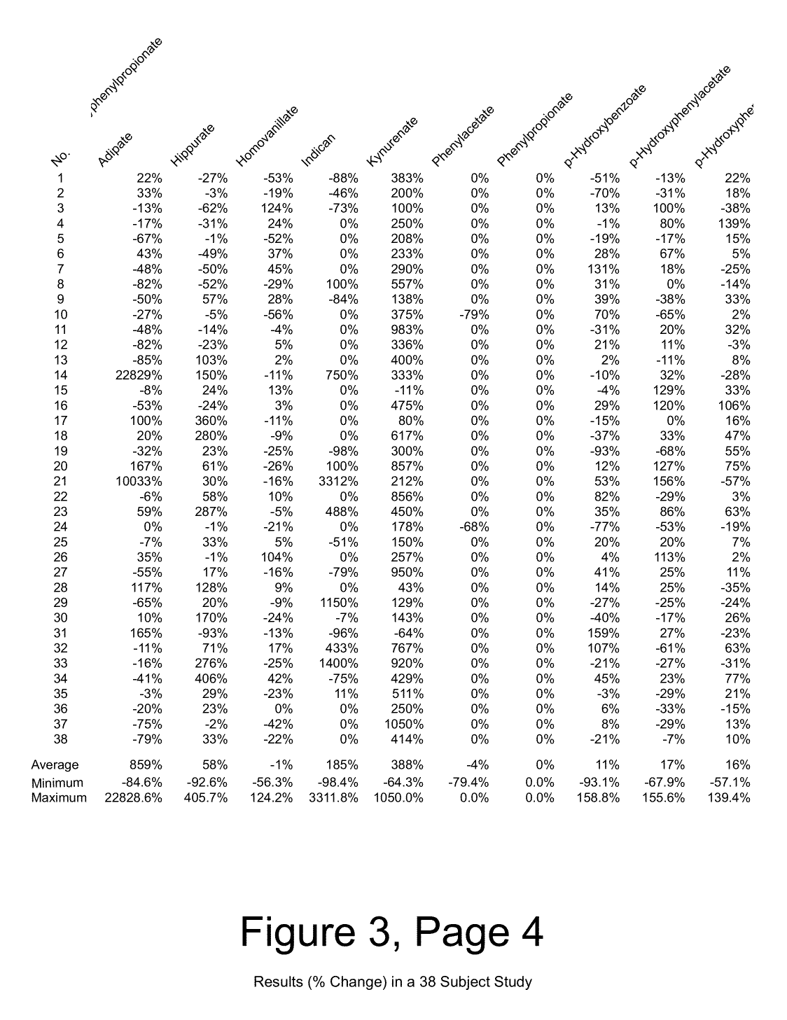

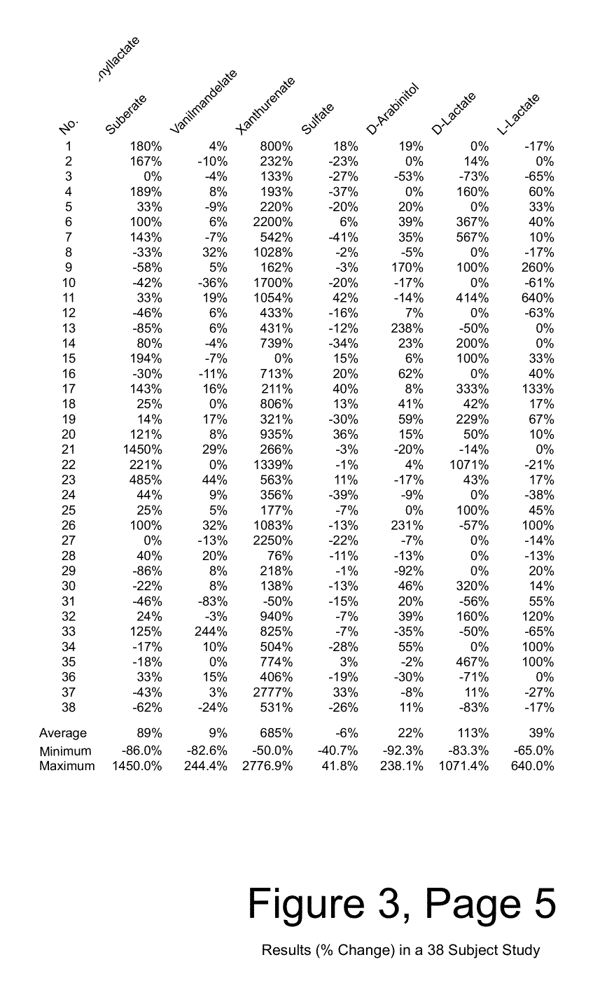

FIG. 3 shows the results of the 38 person study for all analytes. A significant change in pre vs. post-challenge levels is defined as a change of greater than two times the reported biological variability.[10] The most consistent significant increases across all subjects were found for the primary marker compounds derived from amino acids in the challenge formula. The largest overall change among the expected markers listed in Table 1 was the 10,150% increase found for FIGLU (Average=1697%). At least one subject showed a significant change for every analyte except for pyroglutamic, sulfuric, phenylacetic and phenylpropionic acids.

Four subjects had extreme changes for one or more analytes. The areas where these subjects displayed large effects included some of the compounds directly related to the challenge amino acids (formiminoglutamic, xanthurenic, kynurenic acids). Other compounds not found in the amino acid catabolic pathways that were extremely affected in some subjects were creatinine and adipic, suberic, 2-ketoglutaric and glutaric acids, and indoxyl sulfate.

Significant changes in analytes not found in the catabolic pathways for any of the amino acids in the challenge formula demonstrate the ability of the procedure to reveal biochemical individualities due to mechanisms other than simple substrate push in known catabolic sequences.

The areas involved in these exceptional mechanisms include:
1 Mitochondrial function
   a. Up to 10,000% stimulation of adipic acid excretion
   b. Up to 1,450% stimulation of suberic acid excretion
   c. Up to 304% stimulation of citric acid excretion
   d. Up to 75% stimulation of cis-aconitic acid excretion
   e. Up to 55% stimulation of isocitric acid excretion
   f. Up to 2,411% stimulation of 2-ketoglutaric acid excretion
   g. Up to 131% stimulation of succinic acid excretion
   h. Up to 500% stimulation of fumaric acid excretion
   i. Up to 3,067% stimulation of malic acid excretion
   j. Up to 156% stimulation of 3-hydroxybutyric acid excretion
2 Steroid biosynthesis
   k. Up to 100% stimulation of 3-hydroxy-3-methylglutaric acid excretion
3 Stress hormone and neurotransmitter production
   l. Up to 83% decrease in norepinephrine turnover (homovanillic acid excretion)
   m. Up to 104% decrease in epinephrine turnover (vanilmandelic acid excretion)
   n. Up to 164% increase in serotonin turnover (5-hydroxyindoleacetic acid excretion)
4 Cell mitosis regulation
   o. Up to 139% stimulation of p-hydroxyphenyllactic acid excretion
5 Oxidative stress
   p. Up to 64% stimulation of 8-hydroxy-3′-deoxyguanine excretion
6 Detoxification
   q. Up to 602% stimulation of 2-methylhippuric acid excretion (xylene mobilization)
   r. Up to 763% stimulation of orotic acid excretion (ammonia stress)
   s. Up to 729% stimulation of glucarate excretion (phase I & II biotransformation)
   t. Up to 406% stimulation in hippuric acid excretion (glycine conjugation)
   u. Up to 200% stimulation of 2-hydroxybutyric acid excretion (hepatic glutathione biosynthesis)
7 Intestinal bacterial overgrowth
   v. Up to 3312% stimulation in indoxyl sulfate excretion
   w. Up to 3329% stimulation in benzoate excretion
   x. Up to 1071% stimulation in D-lactic acid excretion
   y. Up to 238% stimulation in arabinitol excretion
   z. Up to 545% stimulation in 3,4-dihydroxyphenylpropionic acid excretion

The principal goal of the study was to determine whether the dosages used were sufficient to produce significant increases in organic acid analytes known to be biomarkers of specific nutrient deficiencies. Since the specimens were collected during two successive nights, the biological variabilities for each analyte provide a basis for claiming significance of any changes found. In the prior art, those values have been reported for urine specimens collected once a week for eight consecutive weeks. FIGS. 2 and 3 show the range of elevations found in this study along with the biological variabilities of the measured analytes.

Although the specific pathway intermediates or products described in the methods sections and specified in Table 1 were chosen for analysis in these studies, the challenge procedure would cause increased production of the other intermediates of the pathways. These compounds could also be chosen for diagnosis of metabolic disorders, especially in individuals with inherited metabolic diseases that manifest due to defects in the enzymes required for clearing them. These compounds include, for isoleucine, 2-methylbutyric aid, 2-methyl-3-hydroxybutyric acid, 2-methylacetoacetic acid, methylmalonic acid and propionic acid. Compounds in the leucine pathways include isovaleric acid, 3-methylcrotonic acid, 3-methylglutaconic acid, 3-hydroxy-3-methylglutaric acid, mevalonic acid, squalene, lanosterol and cholesterol. Compounds in the valine pathways include isobutyric acid and methacrylic acid. Compounds in the histidine pathways include histamine, N-methylhistamine, urocanic acid, 4-imidazolone-5-propionic acid and hydantoin propionic acid. Compounds in the tryptophan pathways include 5-hydroxytryptophan, serotonin, melatonin, bufotenine, indolepyruvic acid, indole acetaldehyde, tryptamine, indole acetaldehyde, indoleacetic acid, N-formylkynurenin, kynurenin, 3-hydroxykynurenin, 3-hydroxyanthranilic acid, 2-aminomucononic acid semialdehyde, 2-aminomuconic acid, nicotinic acid, nicotinate nucleotide, nicotinamide adenine dinucleotide and nicotinamide adenine dinucleotide phosphate. Compounds in the tyrosine pathways include monoiodotyrosine, diiodotyrosine, 3,5,3′-triiodotyrosine, thyroxine, dihydroxyphenylalanine, dopaquinone, melanin, dopamine, norepinephrine and epinephrine. Compounds in the alanine (pyruvate) pathway include oxaloacetate, reduced nicotinamide adenine dinucleotide, reduced nicotinamide adenine dinucleotide phosphate, reduced flavin mononucleotide, reduced flavin adenine dinucleotide and glucose and blood lipids.

The doses used in the present invention are sufficient to challenge multiple pathways leading to production of the measured organic acids and achieve improvements in the routine clinical application of urinary organic acid profiling. In addition, by combining multiple amino acids, synergistic effects are exposed due to multiple bio-chemical interactions. For example, the rise in xanthurenic acid due to deficiency of vitamin B6 would be exacerbated by simultaneously increasing demand for pyridoxal-5-phosphate due to greater activities for multiple other transaminase enzymes.

The capacity of an individual to maintain normal urinary excretions of metabolic intermediates under the increased flux conditions created by the amino acid challenge dose determines the degree of increase produced by the challenge. Even mild insufficiencies or enzyme abnormalities that might be missed under non-challenged conditions may become apparent under conditions of the challenge. Individuals with excellent nutrient repletion status are revealed by the fact that no elevations of organic acids are found, even though the pathways were challenged to process higher levels of amino acid substrates and metabolic pathway intermediates.

All of the compositions, processes and methods disclosed and claimed herein can be made and executed without undue experimentation in light of the present disclosure. While the compositions, processes and methods of this invention have been described in terms of preferred embodiments, it will be apparent to those of skill in the art that variations may be applied to the compositions, processes and methods and in the steps or in the sequence of steps of the methods described herein without departing from the concept, spirit, and scope of the invention.

More specifically, it will be apparent that certain compositions, such as amino acid compositions, which are both chemically and physiologically related may be substituted for the compositions described herein while the same or similar results would be achieved. All such similar substitutes and modifications apparent to those skilled in the art are deemed to be within the spirit, scope, and concept of the invention.

What is claimed is:

1. A process for detecting and evaluating metabolic disturbances and specific nutrient insufficiencies within an individual wherein:
the individual consumes an oral amino acid challenge supplement comprising at least two of the amino acids selected from the group: L-Valine, L-Leucine, L-Isoleucine, L-Tryptophan, L-Histidine, and L-Alanine;
a urinary sample is collected from the individual after consuming the oral amino acid supplement; and
a urinary organic acid profile of the urinary sample is evaluated, wherein the levels of metabolic markers within the organic acid profile are evaluated to determine the presence of metabolic disturbances or nutrient insufficiencies within the individual.

2. The urinary organic acid profile of claim 1, wherein the levels of metabolic markers within the urinary sample are evaluated to determine at least one of genetic impairments, toxicant impairments, mitochondrial function impairment, or small intestinal dysbiosis.

3. The urinary organic acid profile of claim 1, wherein the individual is challenged with the oral amino acid challenge supplement at least 8 hours prior to collection of the urinary sample.

4. The urinary organic acid profile of claim 1, wherein the individual is challenged with the oral amino acid challenge supplement prior to 11 PM and the collection of the urinary sample occurring after 7 AM of the following morning.

5. The urinary organic acid profile of claim 1, wherein when the individual weighs between 100 to 200 lbs, the dosage of the amino acids within the oral amino acid challenge supplement comprise at least one of: 2.4-3.6 grams L-Valine, 3.1-4.7 grams L-Leucine, 3.1-4.7 grams L-Isoleucine, 1.2-1.8 grams L-Tryptophan, 3.6-5.4 grams L-Histidine, or 2.4-3.6 grams L-Alanine.

6. The urinary organic acid profile of claim 1, wherein when the individual weighs between 100 to 200 lbs, the dosage of the amino acids within the oral amino acid challenge supplement comprise at least one of: 3.0 grams L-Valine, 3.9 grams L-Leucine, 3.9 grams L-Isoleucine, 1.5 grams L-Tryptophan, 4.5 grams L-Histidine, or 3.0 grams L-Alanine.

7. The urinary organic acid profile of claim 1, wherein when the individual weighs more than 200 lbs, the dosage of the amino acids within the oral amino acid challenge supplement comprise at least one of: 3.2-4.8 grams L-Valine, 4.1-6.2 grams L-Leucine, 4.1-6.2 grams L-Isoleucine, 1.6-2.4 grams L-Tryptophan, 4.8-7.2 grams L-Histidine, or 3.2-4.8 grams L-Alanine.

8. The urinary organic acid profile of claim 1, wherein when the individual weighs more than 200 lbs, the dosage of the amino acids within the oral amino acid challenge supplement comprise at least one of: 4.0 grams L-Valine, 5.2 grams L-Leucine, 5.2 grams L-Isoleucine, 2.0 grams L-Tryptophan, 6.0 grams L-Histidine, or 4.0 grams L-Alanine.

9. The urinary organic acid profile of claim 1, wherein when the individual weighs between 50 to 100 lbs, the dosage of the amino acids within the oral amino acid challenge supplement comprise at least one of: 1.6-2.4 grams L-Valine, 2.1-3.1 grams L-Leucine, 2.1-3.1 grams L-Isoleucine, 0.8-1.2 grams L-Tryptophan, 2.4-3.6 grams L-Histidine, or 1.6-2.4 grams L-Alanine.

10. The urinary organic acid profile of claim 1, wherein when the individual weighs between 50 to 100 lbs, the dosage of the amino acids within the oral amino acid challenge supplement comprise at least one of: 2.0 grams L-Valine, 2.6 grams L-Leucine, 2.6 grams L-Isoleucine, 1.0 grams L-Tryptophan, 3.0 grams L-Histidine, or 2.0 grams L-Alanine.

11. An oral amino acid challenge supplement wherein:
the oral amino acid challenge supplement comprises at least two of the amino acids selected from the group: L-Valine, L-Leucine, L-Isoleucine, L-Tryptophan, L-Histidine, and L-Alanine;
the oral amino acid challenge supplement is consumed by an individual and processed by the individuals biochemical pathways prior to a urine sample collection; and
wherein the urine sample collected is used to evaluate a urinary organic acid profile, the levels of metabolic markers within the urinary organic acid profile are evaluated to determine the presence of metabolic disturbances or nutrient insufficiencies within the individual.
